(19) United States
(12) Patent Application Publication
(10) Pub. No.: US 2003/0017126 A1
(43) Pub. Date: Jan. 23, 2003

(54) Hair treatment compositions

(75) Inventors: Anand Ramchandra Mahadeshwar, Bebington (GB); Ruby Loo Tan-Walker, Bebington (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/192,070

(22) Filed: Jul. 10, 2002

(30) Foreign Application Priority Data
Jul. 11, 2001 (EP) 01305980.3

(51) Int. Cl. A61K 7/06

(52) U.S. Cl. 424/70.1

(57) ABSTRACT

The present invention describes an aqueous washing composition comprising (i) from 0.05 to 5 wt % cationic surfactant containing a hydrocarbyl chain of length from 8 to 22 carbon atoms, (ii) an anionic surfactant containing a hydrocarbyl chain of length from 8 to 22 carbon atoms which is within 2 carbons atoms of the length of the hydrocarbyl chain of the cationic surfactant (i), the weight ratio of anionic surfactant (ii) to cationic surfactant (i) being greater than 1:1, (iii) a deposition aid, and (iv) water-insoluble particles which are anionic or nonionic in nature, the weight ratio of (iv) to (i) being in the range from 150:1 to 2:3.

### FIELD OF THE INVENTION

This invention relates to a washing composition for washing a surface to deposit thereon water-insoluble particles.

### BACKGROUND AND PRIOR ART

In addition to washing or cleansing, it is often beneficial for washing compositions, such as for example shampoos, shower gels and facial washes, to deposit a particulate material on the skin or hair to provide a cosmetic and/or therapeutic benefit. For example, shampoos can contain hair conditioning agents, such as oils and silicones, and solid antimicrobial agents. In order for these agents to work, they have to deposit effectively and in sufficient quantity onto the surface to which they are applied, i.e. onto the hair and/or scalp.

The use of deposition aids in washing compositions is well-known. For example, cationic deposition polymers are widely used in shampoo and conditioner formulations to aid deposition of water-insoluble particulate conditioning agents such as silicone oil droplets. See for example EP 093,601 and EP 529,883 (Unilever).

The use of cationic surfactants in washing compositions, especially hair treatment compositions, is also known. In hair treatment compositions, cationic surfactants are typically employed for their conditioning properties.

However, in washing compositions which contain cleansing anionic surfactant and cationic surfactant compatibility problems can occur. For example, in hair shampoos, cationic surfactant can complex with anionic surfactant resulting in poor deposition and poor conditioning in a shampoo context.

Surprisingly and advantageously, the present inventors have found that in the case of water-insoluble particles which are anionic or nonionic in nature, washing compositions which contain a combination of anionic surfactant, cationic surfactant, cationic deposition aid and the water-insoluble particles provide significantly improved deposition of the particles onto the surface being washed when the hydrocarbyl chain lengths of the anionic and cationic surfactants are similar and the ratio of cationic surfactant to the water-insoluble particles is controlled within defined parameters.

### SUMMARY OF THE INVENTION

This invention provides an aqueous washing composition comprising:

(i) from 0.05 to 5% by weight of a cationic surfactant containing a hydrocarbyl chain of length from 8 to 22 carbon atoms;

(ii) an anionic surfactant containing a hydrocarbyl chain of length from 8 to 22 carbon atoms which is within 2 carbons atoms of the length of the hydrocarbyl chain of the cationic surfactant (i), the weight ratio of anionic surfactant (ii) to cationic surfactant (i) being greater than 1:1;

(iii) a deposition aid; and

(iv) water-insoluble particles which are anionic or nonionic in nature,

in which the weight ratio of the water-insoluble particles (iv) to the cationic surfactant (i) is in the range from 150:1 to 2:3.

### DETAILED DESCRIPTION OF THE INVENTION

Product Form

Washing compositions in which the present invention may be employed will be evident to the skilled person and include compositions for topical application to the body, i.e. to the skin, hair or teeth, as well as laundry and hard surface cleaning compositions. Suitable examples of compositions for topical application include hair treatment compositions, facial washes and shower gels. In all cases the washing composition must be aqueous and work on the basis of dilution with water, i.e. in proper use it is diluted with water.

In a preferred embodiment, the washing composition is a hair treatment composition, more specifically a shampoo. Preferred water-insoluble particles present in hair treatment compositions are particles of an anti-microbial agent, particularly a pyridinethione salt, especially zinc pyridinethione or zirconium pyridinethione, and particles of a water-insoluble oil having hair conditioning attributes, such oil being emulsified in the composition with the aid of suitable emulsifying agents.

The washing composition of the invention may also be one suitable for the washing of fabrics, where the aqueous washing composition comprises particles of a fabric conditioning or treating agent, for example an acrylic latex; or for the washing of hard-surfaces where the aqueous cleaning composition may comprise a germicide, as in, for example, compositions for the cleaning of toilets, or it may comprise a polymer latex designed to leave a polymer film on the cleaned surface, for example to provide a glossy appearance to the surface, or it may comprise a perfume oil.

The washing composition of the invention may also find application in the field of oral hygiene where the deposition during use of an oral treatment composition of particles consisting of or comprising an active compound for the care of the oral cavity, including the teeth, may be beneficial.

Component (i): Cationic Surfactant

Washing compositions according to the invention comprise from 0.05 to 5% by weight of a cationic surfactant containing a hydrocarbyl chain of length from 8 to 22 carbon atoms.

If the washing composition is for topical application, then the cationic surfactant must be cosmetically acceptable and suitable for topical application.

Cationic surfactants useful in compositions of the invention include those containing amino or quaternary ammonium hydrophilic moieties which are positively charged when dissolved in the aqueous composition of the present invention.

Examples of suitable cationic surfactants are those corresponding to the general formula:

[N(R₁)(R₂)(R₃)(R₄)]⁺(X)⁻

in which R₁, R₂, R₃, and R₄ are independently selected from (a) an aliphatic group of from 1 to 22 carbon atoms, or (b) an aromatic, alkoxy, polyoxyalkylene, alkylamido, hydroxyalkyl, aryl or alkylaryl group having up to 22 carbon atoms; and X is a salt-forming anion such as those selected from halogen, (e.g. chloride, bromide), acetate, citrate, lactate, glycolate, phosphate nitrate, sulphate, and alkylsulphate radicals.

The aliphatic groups can contain, in addition to carbon and hydrogen atoms, ether linkages, and other groups such as amino groups. The longer chain aliphatic groups, e.g., those of about 12 carbons, or higher, can be saturated or unsaturated.

The most preferred cationic surfactants for washing compositions, e.g. shampoo compositions, of the present invention are monoalkyl quaternary ammonium compounds in which the alkyl chain length is from C8 to C22, preferably from C8 to C14.

Suitable examples of such materials correspond to the general formula:

[N(R₅)(R₆)(R₇)(R₈)]⁺(X)⁻

in which R₅ is a hydrocarbyl chain having 8 to 14 carbon atoms or a functionalised hydrocarbyl chain with 8 to 14 carbon atoms and containing ether, ester, amido or amino moieties present as substituents or as linkages in the radical chain, and R₆, R₇ and R₈ are independently selected from (a) hydrocarbyl chains of from 1 to about 4 carbon atoms, or (b) functionalised hydrocarbyl chains having from 1 to about 4 carbon atoms and containing one or more aromatic, ether, ester, amido or amino moieties present as substituents or as linkages in the radical chain, and X is a salt-forming anion such as those selected from halogen, (e.g. chloride, bromide), acetate, citrate, lactate, glycolate, phosphate nitrate, sulphate, and alkylsulphate radicals.

The functionalised hydrocarbyl chains (b) may suitably contain one or more hydrophilic moieties selected from alkoxy (preferably C₁-C₃ alkoxy), polyoxyalkylene (preferably C₁-C₃ polyoxyalkylene), alkylamido, hydroxyalkyl, alkylester, and combinations thereof.

Preferably the hydrocarbyl chains R₁ have 12 to 14 carbon atoms, most preferably 12 carbon atoms. They may be derived from source oils which contain substantial amounts of fatty acids having the desired hydrocarbyl chain length. For example, the fatty acids from palm kernel oil or coconut oil can be used as a source of C8 to C12 hydrocarbyl chains.

Typical monoalkyl quaternary ammonium compounds of the above general formula for use in washing compositions of the invention include:

(i) lauryl trimethylammonium chloride (available commercially as Arquad C35 ex-Akzo); cocodimethyl benzyl ammonium chloride (available commercially as Arquad DMCB-80 ex-Akzo)

(ii) compounds of the general formula:

[N(R₁)(R₂)((CH₂CH₂O)ₓH)((CH₂CH₂O)_{y}H)]⁺(X)⁻

in which:

x+y is an integer from 2 to 20;

R₁ is a hydrocarbyl chain having 8 to 14, preferably 12 to 14, most preferably 12 carbon atoms or a functionalised hydrocarbyl chain with 8 to 14, preferably 12 to 14, most preferably 12 carbon atoms and containing ether, ester, amido or amino moieties present as substituents or as linkages in the radical chain;

R₂ is a C₁-C₃ alkyl group or benzyl group, preferably methyl, and

X is a salt-forming anion such as those selected from halogen, (e.g. chloride, bromide), acetate, citrate, lactate, glycolate, phosphate nitrate, sulphate, methosulphate and alkylsulphate radicals.

Suitable examples are PEG-n lauryl ammonium chlorides (where n is the PEG chain length), such as PEG-2 cocomonium chloride (available commercially as Ethoquad C12 ex-Akzo Nobel); PEG-2 cocobenzyl ammonium chloride (available commercially as Ethoquad CB/12 ex-Akzo Nobel); PEG-5 cocomonium methosulphate (available commercially as Rewoquat CPEM ex-Rewo); PEG-15 cocomonium chloride (available commercially as Ethoquad C/25 ex-Akzo)

(iii) compounds of the general formula:

[N(R₁)(R₂)(R₃)((CH₂)ₙOH)]⁺(X)⁻

in which:

n is an integer from 1 to 4, preferably 2;

R₁ is a hydrocarbyl chain having 8 to 14, preferably 12 to 14, most preferably 12 carbon atoms;

R₂ and R₃ are independently selected from C₁-C₃ alkyl groups, and are preferably methyl, and

X is a salt-forming anion such as those selected from halogen, (e.g. chloride, bromide), acetate, citrate, lactate, glycolate, phosphate nitrate, sulphate, and alkylsulphate radicals.

Suitable examples are lauryldimethylhydroxyethylammonium chloride (available commercially as Prapagen HY ex-Clariant).

The total amount of the cationic surfactant in washing compositions of the invention (including any which may be present as emulsifier for hydrophobic ingredients of the shampoo composition) is generally from 0.05 to 5%, preferably from 0.05 to 3%, more preferably from 0.1% to 2.5%, most preferably from 0.1 to 2% by weight based on total weight of the washing composition.

In a preferred embodiment, the cationic surfactant (i) and anionic surfactant (ii) contain hydrocarbyl chains of the same length.

In the case of quaternary ammonium cationic surfactants, it is not necessary, in the case of, for example, a dialkyl quat, for both hydrocarbyl chains to have from 8 to 22 carbon atoms or to be of the same length. However, it is preferable that they are substantially the same length.

In a preferred embodiment, the anionic surfactant is sodium lauryl ether sulphate and the cationic surfactant is a C₁₂ monoalkyl quat.

Component (ii): Anionic Surfactant

Washing compositions according to the invention comprise a cleansing anionic surfactant (ii) which satisfies the following criteria:

it contains a hydrocarbyl chain of length of from 8 to 22 carbon atoms.

the length of the hydrocarbyl chain is within 2 carbons atoms of the length of the hydrocarbyl chain of the cationic surfactant (ie. the length of the hydrocarbyl chain is the same as that of the cationic, 2 less or 2 more.)

the weight the weight ratio of the anionic surfactant (ii) to cationic surfactant (i) in the washing composition is greater than 1:1;

Preferably, the weight ratio of anionic surfactant (ii) to cationic surfactant (i) in the washing composition is at least 1.5:1, more preferably at least 2:1.

If the washing composition is for topical application, then the anionic surfactant must be cosmetically acceptable and suitable for topical application.

Examples of suitable anionic surfactants are the alkyl sulphates, alkyl ether sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates, and alpha-olefin sulphonates, especially their sodium, magnesium, ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from 1 to 10 ethylene oxide or propylene oxide units per molecule.

Typical anionic surfactants for use in compositions of the invention include sodium oleyl succinate, ammonium lauryl sulphosuccinate, ammonium lauryl sulphate, sodium dodecylbenzene sulphonate, triethanolamine dodecylbenzene sulphonate, sodium cocoyl isethionate, sodium lauryl isethionate and sodium N-lauryl sarcosinate. The most preferred anionic surfactants are sodium lauryl sulphate, sodium lauryl ether sulphate, sodium lauryl ether sulphate(n)EO (where n ranges from 1 to 5), ammonium lauryl sulphate and ammonium lauryl ether sulphate(n)EO, (where n ranges from 1 to 3).

More than one anionic surfactant may be present in the compositions of the invention, so long as at least one surfactant satisfies all three of the above criteria. Typically, any other anionic surfactant(s) present in the washing compositions of the invention is also selected from those mentioned above, although it does not necessarily have to satisfy all three criteria.

Suitably, the total amount of anionic surfactant in the washing composition of the invention (including any which may be present as emulsifier for hydrophobic ingredients of the washing composition) is at least 2% by weight, more preferably at least 5% by weight. Suitably, the amount of anionic surfactant ranges from 5 to 30%, preferably from 6 to 24%, more preferably from 8% to 20% by weight based on total weight of the washing composition. These figures apply to equally to compositions containing one and mixtures of anionic surfactants.

Component (iii): Deposition Aid

Washing compositions according to the invention comprise a deposition aid, preferably a cationic deposition polymer.

The cationic polymer may be a homopolymer or be formed from two or more types of monomers. The molecular weight of the polymer will generally be between 5 000 and 10 000 000 Daltons, typically at least 10 000 and preferably in the range 100 000 to about 2 000 000. The polymers will have cationic nitrogen-containing groups such as quaternary ammonium or protonated amino groups, or a mixture thereof. Suitably, the cationic charge density is at least 0.1 meq/g, preferably at least 0.8 or higher. The cationic charge density should not exceed 4 meq/g, it is preferably less than 3 and more preferably less than 2 meq/g. The charge density can be measured using the Kjeldahl method and should be within the above limits at the desired pH of use, which will in general be from about 3 to 9 and preferably between 4 and 8.

The cationic nitrogen-containing group will generally be present as a substituent on a fraction of the total monomer units of the cationic polymer. Thus when the polymer is not a homopolymer it can contain spacer non-cationic monomer units. Such polymers are described in the CTFA Cosmetic Ingredient Directory, 3rd edition. The ratio of the cationic to non-cationic monomer units is selected to give a polymer having a cationic charge density in the required range.

Suitable cationic polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general secondary and tertiary amines, especially tertiary, are preferred.

Amine substituted vinyl monomers and amines can be polymerised in the amine form and then converted to ammonium by quaternization.

The cationic polymers can comprise mixtures of monomer units derived from amine- and/or quaternary ammonium-substituted monomer and/or compatible spacer monomers.

Suitable cationic polymers include, for example:

copolymers of 1-vinyl-2-pyrrolidine and 1-vinyl-3-methyl-imidazolium salt (e.g. chloride salt), referred to in the industry by the Cosmetic, Toiletry, and Fragrance Association, (CTFA) as Polyquaternium-16. This material is commercially available from BASF Wyandotte Corp. (Parsippany, N.J., USA) under the LUVIQUAT tradename (e.g. LUVIQUAT FC 370);

copolymers of 1-vinyl-2-pyrrolidine and dimethylaminoethyl methacrylate, referred to in the industry (CTFA) as Polyquaternium-11. This material is available commercially from GAF Corporation (Wayne, N.J., USA) under the GAFQUAT tradename (e.g., GAFQUAT 755N);

cationic diallyl quaternary ammonium-containing polymers including, for example, dimethyldiallylammonium chloride homopolymer and copolymers of acrylamide and dimethyldiallylammonium chloride, referred to in the industry (CTFA) as Polyquaternium 6 and Polyquaternium 7, respectively;

mineral acid salts of amino-alkyl esters of homo-and copolymers of unsaturated carboxylic acids having from 3 to 5 carbon atoms, (as described in U.S. Pat. No. 4,009,256);

cationic polyacrylamides (as described in WO95/22311).

Other cationic polymers that can be used include cationic polysaccharide polymers, such as cationic cellulose derivatives, cationic starch derivatives, and cationic guar gum derivatives.

Cationic polysaccharide polymers suitable for use in compositions of the invention include those of the formula:

A-O—[R—N⁺(R¹)(R²)(R³)X⁻],

wherein: A is an anhydroglucose residual group, such as a starch or cellulose anhydroglucose residual. R is an alkylene, oxyalkylene, polyoxyalkylene, or hydroxyalkylene group, or combination thereof. R¹, R² and R³ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R¹, R² and R³) is preferably about 20 or less, and X is an anionic counterion.

Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

Other suitable cationic polysaccharide polymers include quaternary nitrogen-containing cellulose ethers (e.g. as described in U.S. Pat. No. 3,962,418), and copolymers of etherified cellulose and starch (e.g. as described in U.S. Pat. No. 3,958,581).

A particularly suitable type of cationic polysaccharide polymer that can be used is a cationic guar gum derivative, such as guar hydroxypropyltrimonium chloride (Commercially available from Rhone-Poulenc in their JAGUAR trademark series).

Examples are JAGUAR C13S, which has a low degree of substitution of the cationic groups and high viscosity. JAGUAR C15, having a moderate degree of substitution and a low viscosity, JAGUAR C17, having a high degree of substitution and a high viscosity, JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

Preferably the cationic polymer is selected from cationic cellulose and cationic guar gum derivatives.

Mixtures of any of the foregoing cationic polymers may also be suitable.

The cationic deposition polymer is suitably present in an amount generally ranging from about 0.01 to about 10%, preferably from 0.01 to 5% by weight, more preferably from 0.01 to 1% by weight of the washing composition.

A particularly preferred level of cationic polymer in shampoo compositions of the invention ranges from 0.05 to 0.5% by weight, since this delivers excellent sensory properties.

Component (iv): Water-Insoluble Particles

Washing compositions according to the invention comprise water-insoluble particles which are anionic or nonionic in nature.

By water-insoluble is meant less than 0.01%, preferably less than 0.001% by weight soluble in water at 25° C. Thus it is an essential requirement that the material of the particles should be insoluble or at most only sparingly soluble in water.

Furthermore, the particles should be anionic or nonionic in nature i.e. they are substantially free of any cationic charges or positively charged groups on the external surface. Thus, the present invention may be used to deposit particles which contain cationically charged material as an inner core and a shell comprising material which presents an anionic or nonionic charge externally.

Otherwise, the nature of the water-insoluble particles employed in the aqueous washing composition is not critical and a wide variety of materials can be deposited onto various substrates. Examples of suitable materials include substances such as anti-microbial agents, sunscreens, fabric brighteners, and various substances that create a favourable skin or hair feel after washing, e.g. conditioning agents.

One class of anionic particulate substances that are of special interest are the salts of pyridinethione (also known as 2-pyridinethiol-1-oxide). These are used in antidandruff hair treatment formulations.

Another class of anionic/nonionic particulate substances that are of special interest are the hard particles described in PCT Application No. PCT/GB00/04020. There include silicas, aluminas and colloidal metals. These are used as styling aids in hair treatment compositions.

Yet another class of particulate substances that are of special interest are benefit agents used in personal care compositions, e.g. conditioners used in hair treatment formulations. These include materials such as silicones and other oily liquids.

The water-insoluble particles can be solid, semi-solid or liquid (e.g. oily) in nature. Thus washing compositions of the invention can be dispersions, colloidal suspensions or emulsions. When appropriate, the water-insoluble particles can be incorporated into the washing compositions of the invention as pre-formed emulsions. In particular, pre-formed emulsions may be used to incorporate silicone particles (see below).

Where pre-formed emulsions are used, the cationic surfactant (i) and/or anionic surfactant (ii) of the washing compositions can be used to pre-emulsify the particles. In a preferred embodiment, only anionic surfactant is used in the pre-emulsification step. However, a portion or all the cationic surfactant (i) present in the final washing composition may also be used to pre-emulsify the particles and thus be incorporated into the washing composition via a pre-formed emulsion. However, where some or all the cationic surfactant (i) is used to pre-emulsify, anionic surfactant (i) must also be used in the pre-emulsifying step at a level significantly greater than the cationic surfactant. Where cationic surfactant (i) is used in the pre-emulsification step, the weight ratio of anionic surfactant (ii) to cationic surfactant (i) is preferably at least 2:1, more preferably at least 3:1, for example 5:1.

Suitably, at least part of the anionic surfactant (ii) is not added via the pre-formed emulsion if one is used.

The particles may consist of a single component or a variety of different components. Also, more than one type of particle may be employed (i.e. particles of different materials).

Suitably, particles have a D₅₀ median particle diameter of from about 0.01 to about 20 microns, preferably from about 0.01 to 10 microns. Preferably, the median particle size is less than 5, more preferably less than 2 and yet more preferably less than 1 micron.

In the case of liquid particles present as an emulsion in the washing composition, particle size may be measured by means of a laser light scattering technique, using a 2600D Particle Sizer from Malvern Instruments. The median D₅₀ is based on the particle volume.

The water-insoluble or sparingly water-soluble particles may be employed in the washing composition in an amount of from 0.001 to about 1% by weight.

It is an essential feature of this invention that the weight ratio of the water-insoluble particles (iv) to the cationic surfactant (i) in the washing compositions is in the range from 150:1 to 2:3. Preferably the weight ratio is at least 100:1 and more preferably at least 20:1. Preferably the weight ratio is no more than 5:6, more preferably no more than 1:1, and yet more preferably no more than 5:4.

As regards the level of deposition of the particles onto the substrate being washed, we have found that different water-insoluble particles have different optimal water-insoluble particle to cationic surfactant weight ratios. For example, particles of silicone in a hair treatment composition deposit best when using a silicone to cationic surfactant weight ratio of from 120:1 to about 4:1. As regards ZnPTO deposition, the optimal ratio for deposition appears to be in the range from 20:1 to about 5:4. The optimal ratio for deposition of silica appears to be in the range from 100:7 to about 5:6. Within the general weight range ratio, the optimal or near optimal ratio for any particulate material can be derived by simple, routine experimentation and is well within the capabilities if the skilled person.

Where more than one type of particle is employed, the particles of one type may have a different optimal ratio for deposition than the particles of another type. In such a case, a ratio which gives acceptable deposition for both particles or, if deposition of one type is more important than for the other type, for one particle may be employed.

Silicone

Preferred water-insoluble particles of the washing compositions of the invention, especially in relation to hair treatment compositions (e.g. shampoos), are non-volatile insoluble silicone particles. The silicone is insoluble in the aqueous matrix of the washing composition and so is present in an emulsified form, with the silicone present as dispersed particles. The particles of silicone when deposited on the substrate being washed typically provide a cosmetic benefit, e.g. conditioning effect to the skin or hair.

Suitable silicones include polydiorganosiloxanes, in particular polydimethylsiloxanes which have the CTFA designation dimethicone.

Also suitable for use in compositions of the invention are hydroxyl functional silicones, in particular polydimethyl siloxanes having hydroxyl end groups which have the CTFA designation dimethiconol.

Also suitable for use in compositions of the invention are silicone gums having a slight degree of cross-linking, as are described for example in WO 96/31188. These materials can impart body, volume and stylability to hair, as well as good wet and dry conditioning.

For dimethicone and dimethiconol-type silicones, the viscosity of the silicone itself is typically at least 10,000 mm²sec⁻¹, preferably at least 60,000 mm²sec⁻¹, most preferably at least 500,000 mm²sec⁻¹, ideally at least 1,000,000 mm²sec⁻¹. Preferably the viscosity does not exceed 10⁹ mm²sec⁻¹ for ease of formulation. For amino functional-type silicones, the viscosity of the silicone itself is not particularly critical and can suitably range from about 100 to about 1,000,000 mm²sec⁻¹. All viscosities are measured at 25° C. using a suitable apparatus such as a capillary viscometer.

Emulsified silicones for use in the washing compositions (e.g. shampoos) of the invention will typically have an average silicone particle diameter D_{3,2} in the composition of less than 30, preferably less than 20, more preferably less than 10 microns. In general, reducing the silicone particle size tends to improve conditioning performance. Most preferably the average silicone particle diameter of the emulsified silicone in the composition is less than 2 microns, ideally it ranges from 0.01 to 1 micron. Silicone emulsions having an average silicone particle size of ≦0.15 microns are generally termed microemulsions.

Particle size may be measured by means of a laser light scattering technique, using a 2600D Particle Sizer from Malvern Instruments.

Suitable silicone emulsions for use in the invention are also commercially available in a pre-emulsified form.

Examples of suitable pre-formed emulsions include emulsions DC2-1766, DC2-1784, and microemulsions DC2-1865 and DC2-1870, all available from Dow Corning. These are all emulsions/microemulsions of dimethiconol. Cross-linked silicone gums are also available in a pre-emulsified form, which is advantageous for ease of formulation. A preferred example is the material available from Dow Corning as DC X2-1787, which is an emulsion of cross-linked dimethiconol gum. A further preferred example is the material available from Dow Corning as DC X2-1391, which is a microemulsion of cross-linked dimethiconol gum.

Mixtures of any of the above types of silicone may also be used.

The total amount of silicone incorporated into compositions of the invention depends on the level of conditioning desired and the material used. A preferred amount is from 0.01 to about 10% by weight of the total composition although these limits are not absolute. The lower limit is determined by the minimum level to achieve conditioning and the upper limit by the maximum level to avoid making the hair and/or skin unacceptably greasy.

When the silicone is incorporated as a pre-formed emulsion as described above, the exact quantity of emulsion will of course depend on the concentration of the emulsion, and should be selected to give the desired quantity of silicone in the final composition.

Optional Ingredients

The washing compositions can optionally include other ingredients. The additional ingredients will vary depending on the nature of the washing composition, but useful additional ingredients will be evident to the skilled person.

For example, additional co-surfactants may be included to help impart aesthetic, physical or cleansing properties to the composition.

A preferred example is an amphoteric or zwitterionic surfactant, which can be included in a total amount (including any which may be present as emulsifier for hydrophobic ingredients of the shampoo composition) ranging from 0 to about 8%, preferably from 1 to 4% by weight based on total weight of the shampoo composition.

Examples of amphoteric and zwitterionic surfactants include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine and preferably lauryl betaine, cocamidopropyl betaine and sodium cocamphopropionate.

Another preferred example is a nonionic surfactant, which can be included in a total amount (including any which may be present as emulsifier for hydrophobic ingredients of the shampoo composition) ranging from 0% to about 8% preferably from 2 to 5% by weight based on total weight of the shampoo composition.

For example, representative nonionic surfactants that can be included in compositions of the invention include condensation products of aliphatic (C₈-C₁₈) primary or secondary linear or branched chain alcohols or phenols with alkylene oxides, usually ethylene oxide and generally having from 6 to 30 ethylene oxide groups.

Other representative nonionic surfactants include mono- or di-alkyl alkanolamides. Examples include coco mono- or di-ethanolamide and coco mono-isopropanolamide.

Further nonionic surfactants which can be included in compositions of the invention are the alkyl polyglycosides (APGs). Typically, the APG is one which comprises an alkyl group connected (optionally via a bridging group) to a block of one or more glycosyl groups. Preferred APGs are defined by the following formula:

RO-(G)ₙ

wherein R is a branched or straight chain alkyl group which may be saturated or unsaturated and G is a saccharide group.

R may represent a mean alkyl chain length of from about C₅ to about C₂₀. Preferably R represents a mean alkyl chain length of from about C₈ to about C₁₂. Most preferably the value of R lies between about 9.5 and about 10.5. G may be selected from C₅ or C₆ monosaccharide residues, and is preferably a glucoside. G may be selected from the group comprising glucose, xylose, lactose, fructose, mannose and derivatives thereof. Preferably G is glucose.

The degree of polymerisation, n, may have a value of from about 1 to about 10 or more. Preferably, the value of n lies in the range of from about 1.1 to about 2. Most preferably the value of n lies in the range of from about 1.3 to about 1.5.

Suitable alkyl polyglycosides for use in the invention are commercially available and include for example those materials identified as: Oramix NS10 ex Seppic; Plantaren 1200 and Plantaren 2000 ex Henkel.

Other sugar-derived nonionic surfactants which can be included in compositions of the invention include the C₁₀-C₁₈ N-alkyl (C₁-C₆) polyhydroxy fatty acid amides, such as the C₁₂-C₁₈-methyl glucamides, as described for example in WO 92 06154 and U.S. Pat. No. 5,194,639, and the N-alkoxy polyhydroxy fatty acid amides, such as C₁₀-C₁₈ N-(3-methoxypropyl) glucamide.

Further Optional Ingredients

Washing compositions of this invention may contain any other ingredients normally used in such washing compositions. For example, a washing composition which is a shampoo may include viscosity modifiers, preservatives, colouring agents, polyols such as glycerine and polypropylene glycol, chelating agents such as EDTA, antioxidants such as vitamin E acetate, fragrances, antimicrobials and sunscreens. Each of these ingredients will be present in an amount effective to accomplish its purpose. Generally these optional ingredients are included individually at a level of up to about 5% by weight of the total composition.

The invention is further illustrated by way of the following non-limitative Examples, in which all percentages quoted are by weight based on total weight unless otherwise stated.

### EXAMPLES

The following base shampoo formulation was prepared:

| Ingredient | wt % |
|---|---|
| Sodium laureth sulphate | 16 |
| Cocoamidopropyl betaine | 2 |
| Jaguar C13S | 0.2 |
| Particulate material | See below |
| Lauryl trimethyl | See below |
| ammonium chloride | |
| Carbopol 980 | 0.4 |
| Formaldehyde | 0.1 |
| Sodium chloride | 1.3 |
| Water | to 100 |

Examples were prepared containing the following particulate materials:

| Example | Particulate material | Charge |
|---|---|---|
| I | 1 wt % silicone (DC1766) | Anionic |
| II | 2 wt % silicone (DC1766) | Anionic |
| III | 2 wt % silicone (DC1766) | Anionic |
| IV | 2 wt % silicone (DC1310) | Nonionic |
| V | 0.25 wt % silica | Anionic |
| VI | 0.5 wt % ZnPTO | Anionic |

DC1766 and DC1310 were supplied by Dow Corning.

For each Example, a series of samples (a-e or a-d) were prepared in which the level of lauryl trimethyl ammonium chloride (i.e. the cationic surfactant) was varied.

All the Examples were tested for their ability to deposit their respective particulate materials onto hair fibres in a hair switch test.

Pre-Wash

0.25 g (5 cm) switches were pre-washed with a 20% soap solution (20 g SLES (27%) in 100 g water) and then with ether. The procedure was repeated twice. After washing the switches were allowed to dry naturally.

Oiling

Except for Examples I and II, the hair switches were oiled prior to being treated with the test shampoo composition. 30 μl of hair oil was applied to a switch and the hair rubbed with the fingers for approximately 30 s. The oiled switches were then left for 30 mins at room temperature.

Test Washes

Each sample (a-e) shampoo formulation was tested in five-fold. Lines of 150 g of shampoo (30 g per switch) were made in 2 petri dishes (one for each wash of sets of 5 switches). Sets of 5 switches were soaked in the petri dish and 1350 μl of water (270 μl per switch) was added. The switches were agitated in the dish for 30 s and rinsed for 30 s under tap water (25° C.) with a flow rate of 3-4 l/min. The procedure was repeated twice. Switches were washed again in the petri dish lid in the same way and allowed to dry naturally.

XFR Analysis

The XRF technique was used to quantify the deposition of active materials on hair fibres.

Cells were soaked in 1.2% SLES 2 EO for 30 mins and then subjected to an ether wash. Double-sided sticky tape was applied to the larger of the two rims, allowing the hair switch to be mounted onto the cell, ensuring all the hair fibres were parallel and covered all the cell, with minimum overlap. The hair fibres were covered with “Chemplex film” (polypropylene X ray film) before being placed in plastic bags for testing.

XRF measures the number of X-rays emitted by all the atoms (e.g. Si or Zn) in the sample when excited by the polychromatic X-rays generated by a Sc-anode tube. The X-ray counts are then related to the active level by constructing a calibration curve as a function of concentration of particulate material.

The level of particulate material deposited (in ppm) as calculated by XFR analysis is given in following table.

| | | | |
|---|---|---|---|
| | Cationic surf. | Wt. ratio of partic. | Deposition |
| Example | (wt %) | to cationic surf. | (ppm) |

Ex I: 1 wt % anionic silicone on non-oiled hair

| | | | |
|---|---|---|---|
| Ia | 0 | — | 275 |
| Ib | 0.025 | 40:1 | 268 |
| Ic | 0.05 | 20:1 | 462 |
| Id | 0.2 | 5:1 | 325 |
| Ie | 0.5 | 2:1 | 346 |

Ex. II: 2 wt % anionic silicone on non-oiled hair

| | | | |
|---|---|---|---|
| IIa | 0 | — | 572 |
| IIb | 0.0175 | 800:7 | 791 |
| IIc | 0.07 | 100:7 | 644 |
| IId | 0.175 | 80:7 | 922 |
| IIe | 0.35 | 40:7 | 950 |

Ex. III: 2 wt % anionic silicone on oiled hair

| | | | |
|---|---|---|---|
| IIIa | 0 | — | 612 |
| IIIb | 0.0175 | 800:7 | 1108 |
| IIIc | 0.07 | 100:7 | 1512 |
| IIId | 0.175 | 80:7 | 1929 |
| IIIe | 0.35 | 40:7 | 1034 |

Ex. IV: 2 wt % nonionic silicone on oiled hair

| | | | |
|---|---|---|---|
| IVa | 0 | — | 442 |
| IVb | 0.05 | 40:1 | 701 |
| IVc | 0.2 | 10:1 | 510 |
| IVd | 0.5 | 4:1 | 486 |
| IVe | 1.0 | 2:1 | 300 |

Ex. V: 0.25 wt % anionic silica on oiled hair

| | | | |
|---|---|---|---|
| Va | 0 | — | 50 |
| Vb | 0.0175 | 100:7 | 68 |
| Vc | 0.175 | 10:7 | 115 |
| Vd | 0.35 | 5:7 | 54 |

Ex. VI: 0.5 wt % anionic ZnPTO on oiled hair

| | | | |
|---|---|---|---|
| VIa | 0 | — | 124 |
| VIb | 0.0315 | 100:6.3 | 169 |
| VIc | 0.07 | 50:7 | 197 |
| VId | 0.175 | 20:7 | 244 |
| VIe | 0.35 | 10:7 | 164 |

What is claimed is:

1. An aqueous washing composition comprising
(i) from 0.05 to 5% by weight of a cationic surfactant containing a hydrocarbyl chain of length from 8 to 22 carbon atoms;
(ii) an anionic surfactant containing a hydrocarbyl chain of length from 8 to 22 carbon atoms which is within 2 carbons atoms of the length of the hydrocarbyl chain of the cationic surfactant (i), the weight ratio of anionic surfactant (ii) to cationic surfactant (i) being greater than 1:1;
(iii) a deposition aid; and
(iv) water-insoluble particles which are anionic or nonionic in nature,
in which the weight ratio of the water-insoluble particles (iv) to the cationic surfactant (i) in the washing compositions is in the range from 150:1 to 2:3.

2. A washing composition according to claim 1, in which the weight ratio of anionic surfactant (ii) to cationic surfactant (i) is at least 1.5:1, more preferably at least 2:1.

3. A washing composition according to claim 1 or claim 2, in which the total amount of anionic surfactant is from 2 to 30% by weight based on the total weight of the washing composition.

4. A washing composition according to claim 1, in which the anionic surfactant is selected from sodium lauryl sulphate, sodium lauryl ether sulphate(n)EO, (where n ranges from 1 to 3), ammonium lauryl sulphate and ammonium lauryl ether sulphate(n)EO, (where n ranges from 1 to 3).

5. A washing composition according to claim 1, in which the cationic surfactant (i) is present from 0.05 to 3%, preferably from 0.1 to 2.5% and more preferably from 0.1 to 2% by weight based on total weight of the washing composition.

6. A washing composition according to claim 1, in which the cationic surfactant (i) is a monoalkyl quaternary ammonium compound in which the alkyl chain length is from C8 to C22, preferably from C8 to C14.

7. A washing composition according to any claim 1, in which the hydrocarbyl chains of the cationic surfactant (i) and the anionic surfactant (ii) are substantially the same length.

8. A washing composition according to any preceding claim 1, in which the water-insoluble particles (iv) comprise silicone.

9. A washing composition according to claim 8, in which the silicone particles are added to the washing composition as a pre-formed emulsion with anionic surfactant.

10. A washing composition according to claim 1, in which the water-insoluble particles (iv) have an D₅₀ volume median diameter of from 0.01 to 20, preferably from 0.01 to 10 microns.

11. A washing composition according to claim 1, in which the weight ratio of the water-insoluble particles (iv) to the cationic surfactant (i) is in the range from 100:1 to 5:6, more preferably from 100:1 to 1:1 and yet more preferably from 20:1 to 5:4.

12. A washing composition according to claim 1, in which the deposition aid (iii) is a cationic deposition polymer selected from cationic cellulose, cationic guar gum derivatives and mixtures thereof.
